(19) United States
(12) Patent Application Publication
(10) Pub. No.: US 2008/0050735 A1
(43) Pub. Date: Feb. 28, 2008

(54) Nucleic acid testing method for point-of-care diagnostics and genetic self-monitoring

(76) Inventor: Elena Pushnova, Oakland, CA (US)

Correspondence Address: ELENA PUSHNOVA, 6949 PINEHAVEN ROAD, OAKLAND, CA, 94611-1017, US

(21) Appl. No.: 11/702,028

(22) Filed: Feb. 1, 2007

Related U.S. Application Data
(60) Provisional application No. 60/763,954, filed on Feb. 1, 2006.

(51) Int. Cl. C12Q 1/68

(52) U.S. Cl. 435/6

(57) ABSTRACT

This invention describes a nucleic acid testing procedure in a form of portable device or a test kit for the purposes of clinical genetic testing, infectious disease diagnostics, biodefense, forensic analysis, paternity testing, pet and cattle breeding, food testing, etc. This testing does not include toxic chemicals and is simple enough to be used by an average individual without any special laboratory training. The procedure includes collecting the sample, potential isothermal amplification of the whole genomic DNA or a fragment of genomic DNA, denaturing double-stranded DNA into single-stranded form, hybridizing the denatured sample DNA to single-stranded allele-specific tester oligonucleotides complementary to the analyzed DNA sequence of interest, selective removal of single-stranded DNA from DNA hybrids, and finally detecting the label in double-stranded hybrids to determine the presence or absence of a particular sequence in the initial sample.

### CROSS-REFERENCE TO RELATED APPLICATIONS

This nonprovisional application claims the benefit and the Feb. 1, 2006 priority date of U.S. Provisional Patent Application Ser. No. 60/763,954, the entire teachings of which are incorporated herein by reference.

### REFERENCES

Sambrook, J. and Russel, D. W. Molecular Cloning: A Laboratory Manual, 3rd edition (2001). Cold Spring Harbor Laboratory Press, Cold Spring Harbor.

Ausubel, F. M. et al. Short Protocols in Molecular Biology, 5th edition (2002). Wiley and Sons, New York, N.Y.

### FIELD OF THE INVENTION

The present invention relates to the area of genetics, molecular biology, molecular diagnostics, and nucleic acid testing. The invention has applications in the fields of clinical genetic testing, infectious disease diagnostics, biodefense, forensic analysis, paternity testing, pet and cattle breeding, food testing, etc.

### STATEMENT REGARDING FEDERALLY SPONSORED RESEARCH OR DEVELOPMENT

Not applicable to this application.

### BACKGROUND OF THE INVENTION

Nucleic Acid Testing is a fast growing area of science and industry, it is used for the purposes of clinical genetic testing, infectious disease diagnostics (including biodefense), human identification (including forensic), paternity and maternity testing, lineage/genealogy determination, pet and cattle breeding, GMO (genetically modified organisms) detection, food testing, etc.

The subject of Nucleic Acid Testing is known SNPs (single nucleotide polymorphisms), InDels (insertions and deletions of one or more nucleotides), and STRs (short tandem repeats) and VNTRs (variable number of tandem repeats) in DNA.

The current state-of-art technologies for nucleic acid testing like RFLP (restriction fragment length polymorphism), SSCP (Single Strand Conformation Polymorphism), DGGE (Denaturing Gradient Gel Electrophoresis), ASO (allele-specific oligonucleotides) and numerous others are highly sophisticated, laborious and expensive. Because these methods utilize such complex technologies as DNA amplification, restriction, denaturing, agarose and polyacrylamide gel electrophoresis, column chromatography, etc., the abovementioned test can be performed in specialized testing labs by highly skilled personnel.

The need for a simple nucleic acid testing device that could be used by an average individual outside of a specialized laboratory settings have existed for a long time, but has yet not been met. Some companies providing paternity testing and lineage identification services claim that they offer an “at home testing”, whereas in reality all that happens at home is collection of sample in a form of buccal swab, which is then mailed to the company for testing.

There have been certain advancements towards development of a point-of-care (e.g. in the hospital) diagnostic device in the areas of microfluidics and nanotechnology by miniatuarizing and automating known nucleic acid testing methods, however they are still far from reaching the goal. Currently Cepheid is offering their point-of-care GeneXpert® System testing product that includes microfluidics-based cartridges for sample preparation, but its high prices makes it unsuitable for self-diagnostics by an average consumer.

Clondiag has developed a promising point-of-care nucleic acid testing device. This company has simplified the ASO (allele-specific hybridization) microchip technology by attaching microchip to the bottom of microcentrifuge tube for ease of treating microchip that is done by means of centrifugation. But the Clondiag product is also unacceptable for use by an average consumer, because their test procedure requires a centrifuge and a microchip scanner.

### SUMMARY OF THE INVENTION

The above described need in point-of-care and self-executed nucleic acid testing is addressed by the subject of present invention.

A nucleic acid testing procedure described in this invention is primarily designed for detection of known SNPs (single nucleotide polymorphisms) and InDels (insertions and deletions of one or more nucleotides), however it can be also used for identification of STRs (short tandem repeats) and VNTRs (variable number of tandem repeats).

This testing does not include toxic chemicals and is simple enough to be used by an average individual without any special laboratory training. The procedure includes collecting the sample, potential isothermal amplification of the whole genomic DNA or a fragment of genomic DNA, denaturing double-stranded DNA into single-stranded form, hybridizing the denatured sample DNA to single-stranded allele-specific tester oligonucleotides complementary to the analyzed DNA sequence of interest, selective removal of single-stranded DNA from DNA hybrids, and finally detecting the label in double-stranded hybrids to determine the presence or absence of a particular sequence in the initial sample.

### BRIEF DESCRIPTION OF THE DRAWINGS

Not Applicable to the present invention.

### DETAILED DESCRIPTION OF THE INVENTION (PREFERRED EMBODIMENTS)

The physical form and design of the diagnostic kit may not be limited to the forms and designs described as preferred embodiments.

Definition

The term “Tester Oligos” is an abbreviation of Tester Oligonucleotides, and unless stated otherwise, defines a single-stranded 20 nucleotide (nt) long DNA molecule containing one or more labeled nucleotides.

Test Sample

At present time buccal swabbing is the most common means of collecting the sample for nucleic acid testing because it is safe, simple, and inexpensive. Buccal swab is produced by rubbing the inner side is both checks, 30 seconds each, with the tip of the swab. The resulting swab contains enough highly stable buccal cell material for nucleic acid testing.

Other popular sampling types include Guthrie blood spots (also known as Guthrie cards) and hair follicles. Not all of these sampling types are equally suitable for a self-diagnostic test: for example Guthrie blood spots obtained by dispensing few drops of peripheral blood on a piece of filter paper may be hazardous in terms of both collecting and handling the sample. A sample for forensic testing may be any type of material (evidence) that carries enough cells or DNA for genetic identification.

As will follow from the later steps of the procedure, in order to obtain a reliable test result the number of DNA copies in the sample, weither copies of genome or of a specific DNA fragment, must be high enough in hybridization with the tester oligos. Therefore amplification of the sample DNA may be required prior to analysis.

Nucleic acid amplification is the enzymatic synthesis of nucleic acid amplicons (copies) that contain a sequence that is complementary to a nucleic acid sequence being amplified. There are thermocycling and isothermal types of DNA amplification performed varying and constant temperatures, respectively. Examples of nucleic acid amplification procedures practiced in the art include the polymerase chain reaction (PCR), strand displacement amplification (SDA), ligase chain reaction (LCR), and transcription-associated amplification (TAA).

SDA may be preferred type of amplification for a self-diagnostic kit mainly because this amplification is isothermal and therefore does not require an expensive thermocycler. SDA primers are random hexamers, and SDA amplification product represents the whole genome. In current method he primers in SDA must be in limiting concentration, about 10 times lower than in conventional SDA protocol: the latter assures that after amplification all primers will be utilized, and will not interfere with the test by hybridizing to tester oligos.

If amplification is necessary, the buccal swab or other sample is immersed into a solution required for amplification, whether an amplification buffer or amplification denaturing solution.

If the subject of testing contains its genetic information in a form of RNA, a reverse transcription step with an optional subsequent amplification may be desirable to stabilize RNA in the sample by converting it to DNA.

DNA Denaturing

In one embodiment the double-stranded sample DNA is denatured in alkaline conditions like high pH solution of NaOH and SDS. The sample containing cell material or its amplified derivative is mixed with alkaline denaturing solution for lysis of the cells and rendering sample DNA in a single-stranded form.

Other embodiments may include alternative means of DNA denaturing such as by heating. This type of denaturing may be accomplished by incubating either sample DNA suspended in TE (Tris-EDTA) buffer or the amplified sample DNA at about 95° C. for 5-20 minutes. After such heat denaturing the sample must be placed on ice immediately to prevent re-annealing of single-stranded DNA.

Heat denaturing however has certain disadvantages in terms of its use as part of self-diagnostic test. First of all incubation at 95° C is potentially hazardous due to a possibility of burn. Besides heat denaturing requires a heating block to be included in the test kit: that makes the kit more expensive. In addition ice must be provided in the kit with this type of denaturing. Finally, if the sample DNA was amplified prior to denaturing, subsequent alkaline denaturing has an advantage of inactivating any DNA polymerase left in the sample, while heat denaturing may not affect a thermostable enzyme.

Tester Oligos

The regular length of the oligos used for detection of SNPs and InDels is 20 nt. In some embodiments however the test kit may be designed for detection of STR (short tandem repeat) and VNTR (variable number of tandem repeat) sequences where there are several tester oligos, each complementary to a specific number of tandem repeats representing a particular allele of a tandem repeat region.

For detection of a particular single sequence in the sample, as in infectious disease diagnostics, only one tester oligo is required for testing. For the purpose of genotyping (for diploid or polyploid genotype diagnostics, multiplex genotyping, detection of multiple infectious agents, DNA fingerprinting, paternity testing, etc., two or more tester oligonucleotides are used in the test, and the end nucleotide of each sequence-specific or allele-specific tester oligonucleotide is complimentary to a particular polymorphic nucleotide characteristic for each variant or allele of the sequence of interest, and is called a detector nucleotide. The detector nucleotide may be located either at 3′-end or at 5′-end of the tester oligo. Depending on the format of the test, the labels of the detector nucleotides may be same or different, in latter case the sequence-specific or allele-specific labels are called the detector labels.

In one embodiment the detector label is fluorescent label. In another embodiment the detector label is chemiluminescent label. Radioactive labeling although highly sensitive is very hazardous and therefore is not acceptable for a point of-care or self-testing kit, in addition radioactive isotopes are highly unstable. There is another very sensitive type of labeling proprietary to Molecular Devices: the nucleotide is labeled with fluorescein which is in turn detected by binding to a conjugate of anti-fluorescein and urease and subsequent registering a change in the potentiometric signal produced by urease-mediated cleavage of urea; this system however is relatively complex, highly expensive, and therefore is unsuited for use in an over-the-counter diagnostic kit.

Depending on the embodiment, the tester oligos may carry yet another label used for capture of the tester oligos. This capture label is associated with the nucleotide at the end of the oligo that is opposite to the detector nucleotide end. In other words one end of the tester oligo is labeled for capturing, and another end is labeled for detection). An example of a convenient and popular capture label is biotin due to its high-affinity binding to streptavidin.

In some embodiments the tester oligo may be captured after hybridization with denatured sample DNA. Yet in other embodiments the tester oligo may be attached to a carrier such as nitrocellulose, magnetic beads, or any other type of carrier, prior to hybridization with denatured sample DNA Binding of the tester oligo to a carrier may be either direct or via a spacer such as biotin-strepavidin complex or any different type of spacer.

In a different embodiments yet another distinguishable control label may be attached to the nucleotide immediately adjacent to the end detector nucleotide and complimentary to any variant or allele of the sequence of interest in order to control successful hybridization between single-stranded sample DNA and the tester oligonucleotide.

It is desirable to incorporate positive and negative control tester oligonucleotides in the test. These control tester oligos are respectively complimentary to sequences that are known to be present and absent in the sample included in the test. For example, there are no sequences matching those of yeast 2-micron DNA in human genome; thus a tester oligo complementary to a portion of 2 micron DNA FLP gene represents an appropriate negative control oligo for testing in human.

Hybridization.

Following denaturing, the single-stranded sample DNA is hybridized to the labeled oligos. Hybridization is performed in low-stringency conditions. In one embodiment where the tester oligos are in solution, the tester oligos and the denatured sample DNA solutions are combined, mixed, and incubated to allow formation of double-stranded complexes between sample DNA and tester oligos. If the sample DNA has been denatured in alkaline solution, the buffer solution containing tester oligos must have appropriate ionic strength and pH to be able to neutralize the alkaline solution containing denatured sample DNA, and to assure stability of the tester oligos prior to hybridization.

If detector nucleotides of tester oligos are associated with different allele-specific labels, hybridization with both allele-specific oligos occurs in the same tube. However if allele-specific detector nucleotides of the tester oligos have the same label, hybridization of denatured sample DNA with each sequence-specific or allele-specific tester oligonucleotide is performed in a separate reaction.

In other embodiments hybridization occurs between the denatured sample DNA in solution and the tester oligos that are already bound to the carrier. For example the tester oligos may be bound to a strip of nitrocellulose that is in turn attached to the inner surface of a cap that fits the opening of the tube containing denatured sample DNA. Once the tube is closed with such cap, the strip containing tester oligos is immersed into denatured sample DNA solution. If the sample DNA has been denatured in alkaline solution, a neutralization buffer is added to the sample DNA immediately prior to hybridization. In another example the tester oligos are bound via biotin-streptavidin complex to the bottom of the test tube used for hybridization and are provided in the test kit in the neutralizing buffer ready to be combined with the denatured sample DNA.

Post-Hybridization Procedures

Following hybridization, the mixture is subjected to treatment with an agent that is able to remove mispaired nucleotides from nucleic acid strand. Such agent removes the mismatched labeled end detector nucleotides from double-stranded hybrids, as well as digests any excess of tester oligonucleotides and single-stranded sample DNA. This treatment does not remove properly hybridized end detector nucleotides and control nucleotides from double-stranded hybrids, and therefore is crucial for determination of the presence or absence of a specific sequence or allele in the initial test sample.

Usually digestion of single-stranded nucleic acid molecules is accomplished by enzymatic treatment. Enzymes that can be used for this purpose are single-stranded DNA nucleases including Exonuclease I, Mung Bean Nuclease, RecJ, and S1 Nuclease. As an extra precaution after the treatment is complete, these nucleases can be easily inactivated by either addition of EDTA, or slight heating, or combination of both. Another means for digesting single-stranded DNA in hybridization product is by chemical treatment.

In some embodiments the complex formed by the denatured sample DNA and the tester oligos is captured following removal of single-stranded DNA and mismatched nucleotides. For example, the complex containing biotinylated tester oligos may be captured on streptavidin-coated magnetic beads that are added after digestion step.

In the embodiment where the tester oligo was bound to a carrier either before or after hybridization, the double-stranded DNA complex formed by the tester oligos and the sample DNA is washed in low-stringency conditions to remove free labeled nucleotides generated in process of single-stranded DNA digestion. In the abovementioned example where the complex is bound to a strip attached to a tube cap, the strip may be washed either by sequentially transferring the cap with strip into tubes with clean wash solution, or simply by pouring wash solution over the strip. In the second abovementioned example where the complex is bound to magnetic beads, washing is achieved by holding the beads in the tube with a magnet while replacing the solution in the tube with clean wash.

In another embodiment where the complex formed by the tester oligos and the sample DNA is not bound to the carrier, following single-stranded DNA digestion the double-stranded DNA hybrids are separated from free nucleotides in a size-separation medium. For example, the molecules can be separated by size on a small disposable column filled with molecular sieve like gel Sephadex G50: fractionation of molecules in such column may be performed at gravity flow so that at the end the high molecular weight double-stranded DNA hybrid fraction will elute from the column in early void volume fraction, while free nucleotides will be retained in the column. As another option pending advancements in the field of microfluidics a simple in use and reasonably priced capillary gel separation unit may become available for this step.

Registering the Label and Determination of Test Result.

The presence of the tester oligo must be tested easily but reliably: the presence of the label in a particular sequence-specific or allele-specific double-stranded DNA hybrid indicates the presence of the corresponding sequence or allele in the analyzed sample.

The presence of a fluorescent label may be easily registered by using, for example, a simple device represented by a hand-held light source. There are numerous fluororescent labels currently available on the market having fluorophopres that absorb and emit light at different wavelengths.

The presence of label may be also identified using lower sensitivity chromogenic detection by means of binding a-label to an antibody (or in case of biotin label to streptavidin) that is in turn coupled to alkaline phosphatase, with subsequent addition of chromogenic substrate for this enzyme.

What is claimed is:

1. A genetic testing procedure for identification of DNA sequence(s) of interest in the sample that is carried out within a field device or in a test kit format, that is applicable for self-executed diagnostics by any individual, without laboratory settings, toxic chemicals or specific skills being required, and is comprised of:
a) Denaturing double-stranded DNA in the analyzed sample to single-stranded form, where depending on availability of DNA in the sample, genomic DNA may be partially or fully amplified prior to denaturing, and where RNA may be analysed in the same manner as DNA with unstable RNA being converted to DNA by reverse transcription prior to denaturing, and where positive and negative control DNA fragments may be included in the test.
b) Hybridizing denatured DNA with single-stranded sequence-specific or allele-specific tester oligonucleotides complementary to the DNA sequence of interest, where depending on the type of the test, one (e.g. for detection of infectious agents) or more (for diploid or polyploid genotype diagnostics, multiplex genotyping, detection of multiple infectious agents, DNA fingerprinting, paternity testing, etc.) tester oligonucleotides may be used, and where the end nucleotide at either 3′- or 5′-end of the tester oligonucleotide is associated with a label; where if two or more tester oligonucleotides are used in the test, the end nucleotide of each sequence-specific or allele-specific tester oligonucleotide is complimentary to a particular polymorphic nucleotide characteristic for each variant or allele of the sequence of interest and is associated with a label which is unique to each sequence or allele, and where the opposite end of each tester oligonucleotide is modified to allow capture of the oligonucleotide; where in addition to label associated with the end sequence-specific or allele-specific nucleotide of the tester oligonucleotide and therefore called a detector label, a different control label may be attached to the nucleotide immediately adjacent to the end detector nucleotide and complimentary to any variant or allele of the sequence of interest in order to control successful hybridization between single-stranded sample DNA and the tester oligonucleotide; and where positive and negative control tester oligonucleotides complimentary to sequences that are known to be present and absent in the sample, respectively, may be included into the test.
c) Subjecting double-stranded DNA hybrids and possible excess of single-stranded tester oligonucleotides and single-stranded sample DNA to treatment with an agent that cleaves mispaired nucleotides from nucleic acid strand to digest any excess of tester oligonucleotides and single-stranded sample DNA and to remove the mismatched labeled end detector nucleotides from double-stranded hybrids; where properly hybridized end detector nucleotides and control nucleotides are not removed from double-stranded hybrids.
d) Capturing double-stranded hybrids on a carrier.
e) Registering the label present in the captured DNA hybrids, where the presence of a particular sequence-specific or allele-specific label in the captured hybrids indicates the presence of the corresponding sequence or allele in the analyzed sample, and where the presence of the control label indicates successful hybridization between single-stranded sample DNA and the tester oligonucleotide and therefore confirms validity of the result.

2. The procedure of claim 1, wherein the opposite-to-label ends of tester oligonucleotides do not have modifications for oligonucleotide capturing, and in step d) the double-stranded DNA hybrids are separated from free nucleotides after cleavage step c) in a size-separation medium; and wherein the presence of particular label in the higher molecular weight double-stranded DNA hybrid fraction in step e) indicates the presence of the corresponding sequence or allele in the analyzed sample.

3. The procedure of claim 2, wherein sequence-specific or allele-specific tester oligonucleotides have different lengths but have the same label associated with all sequence-specific or allele-specific end nucleotides; and wherein after separating the double-stranded DNA hybrids in a size-separation medium the presence of a particular sequence or allele in the analyzed sample is determined in step e) by presence of the label in a corresponding sequence-specific or allele-specific hybrid size fraction.

4. The procedure of claim 1, wherein hybridization with each sequence-specific or allele-specific tester oligonucleotide and capturing of double-stranded hybrids is performed in a separate reaction, and the end nucleotides of all sequence-specific or allele-specific tester oligonucleotides have the same label; and wherein the presence of the label in a captured hybrid in a particular sequence-specific or allele-specific reaction determined as in step e) indicates the presence of the corresponding sequence or allele in the analyzed sample.

5. The procedure of claim 1, wherein the labeled tester oligonucleotides are captured on the carrier prior to hybridization with denatured sample DNA.

6. The procedure of claim 5, wherein each sequence-specific or allele-specific tester oligonucleotide is attached to a separate carrier, and end nucleotides of all tester oligonucleotides have the same label; and wherein the presence of the label in a particular sequence-specific or allele-specific carrier indicates the presence of the corresponding sequence or allele in the analyzed sample.

7. The procedure of claim 5, wherein each sequence-specific or allele-specific tester oligonucleotide is attached to a designated spot on the same carrier, and end nucleotides of all tester oligonucleotides have the same label; and wherein the presence of the label in a particular sequence-specific or allele-specific spot indicates the presence of the corresponding sequence or allele in the analyzed sample.
